(19) United States
(12) Plant Patent Application Publication
(10) Pub. No.: US 2016/0205848 P1
(43) Pub. Date: Jul. 14, 2016

(54) Hibiscus Plant Named 'Little Zin'

(50) ;

(71) Applicant: Ball Horticultucal Company, West Chicago, IL (US)

(72) Inventor: Scott Trees, Arroyo Grande, CA (US)

(21) Appl. No.: 14/544,486

(22) Filed: Jan. 12, 2015

(51) Int. Cl. A01H 5/00

(52) U.S. Cl. PLT/257

(57) ABSTRACT

A new and distinct cultivar of Hibiscus plant named ‘Little Zin’, characterized by its burgundy colored foliage and moderately vigorous, upright-mounded growth habit, is disclosed.

### LATIN NAME OF GENUS AND SPECIES OF PLANT CLAIMED

Hibiscus acetosella

### VARIETY DENOMINATION

‘Little Zin’

### BACKGROUND OF THE INVENTION

The present invention relates to a new and distinct cultivar of Hibiscus plant botanically known as Hibiscus acetosella and hereinafter referred to by the cultivar name ‘Little Zin’.

The new cultivar originated in a controlled breeding program in Arroyo Grande, Calif. during December 2011. The objective of the breeding program was the development of Hibiscus cultivars with a more compact and well-branched growth habit.

The new Hibiscus cultivar was the result of a self-pollination ‘Mahogany Splendor’, not patented, characterized by its dark burgundy-colored foliage, and vigorous, upright-mounded growth habit. The new cultivar was discovered and selected as a single plant within the progeny of the above stated self-pollination during December 2012 in a controlled environment in Arroyo Grande, Calif.

Asexual reproduction of the new cultivar by terminal stem cuttings since December 2012 in Arroyo Grande, Calif., and West Chicago, Ill. has demonstrated that the new cultivar reproduces true to type with all of the characteristics, as herein described, firmly fixed and retained through successive generations of such asexual propagation.

### SUMMARY OF THE INVENTION

The following characteristics of the new cultivar have been repeatedly observed and can be used to distinguish ‘Little Zin’ as a new and distinct cultivar of Hibiscus plant:

1. Burgundy colored foliage; and

2. Moderately vigorous, upright-mounded growth habit.

Of the many commercially available Hibiscus cultivars, the most similar in comparison to the new cultivar is the parent ‘Mahogany Splendor’, not patented. However, in side by side comparisons, plants of the new cultivar differ from plants of ‘Mahogany Splendor’ in at least the following characteristics:

1. Plants of the new cultivar are shorter than plants of ‘Mahogany Splendor’;

2. Plants of the new cultivar have a crenate leaf margin unlike the serrate leaf margin of plants of ‘Mahogany Splendor’; and

3. Plants of the new cultivar have a lighter foliage color than plants of ‘Mahogany Splendor’.

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs differ slightly from the color values cited in the detailed description, which accurately describes the colors of ‘Little Zin’. The plants were grown in 4-inch pots for 4 weeks then 6-inch pots for 5 weeks in a greenhouse in West Chicago, Ill.

FIG. 1 illustrates a side view of the overall growth and habit of ‘Little Zin’.

FIG. 2 illustrates a close-up view of an individual flower of ‘Little Zin’.

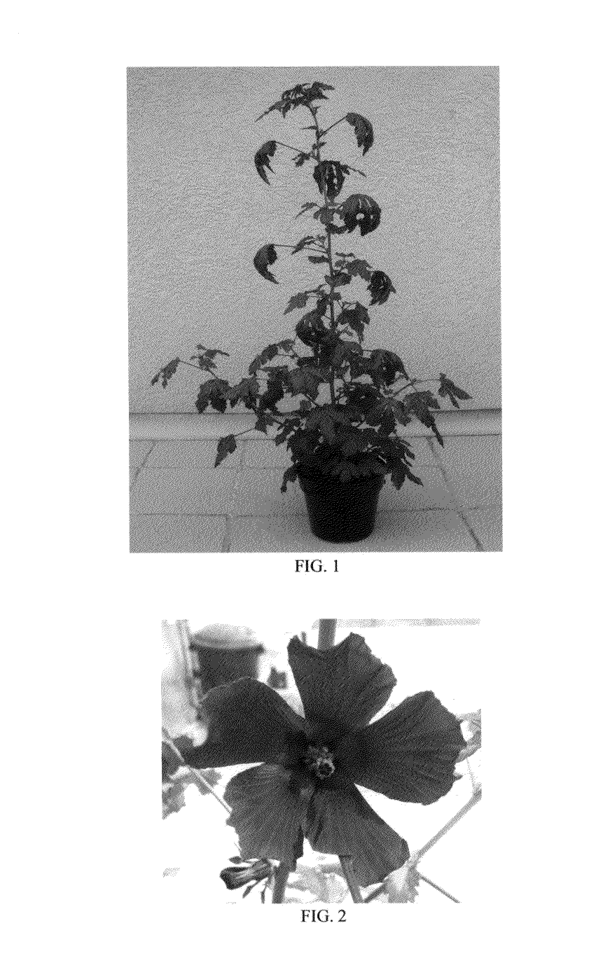

### DETAILED BOTANICAL DESCRIPTION

The new cultivar has not been observed under all possible environmental conditions to date. Accordingly, it is possible that the phenotype may vary somewhat with variations in the environment, such as temperature, light intensity, and day length, without, however, any variance in genotype.

The chart used in the identification of colors described herein is The R.H.S. Colour Chart of The Royal Horticultural Society, London, England, 2007 edition, except where general color terms of ordinary significance are used. The color values were determined in November 2014 under natural light conditions in West Chicago, Ill.

The following descriptions and measurements describe plants produced from cuttings from stock plants and grown in a glass-covered greenhouse under conditions comparable to those used in commercial practice. The plants were grown in West Chicago, Ill. in 4-inch pots for 4 weeks then 6-inch pots for 5 weeks in a greenhouse utilizing a soilless growth medium. Greenhouse temperatures were maintained at approximately 66° F. to 70° F. (19° C. to 21° C.) during the day and approximately 58° F. to 62° F. (14° C. to 17° C.) during the night. Greenhouse light levels of 2,500 footcandles to 6,000 footcandles were maintained during the day. Measurements and numerical values represent averages of typical plants.
Botanical classification: Hibiscus acetosella cultivar Little Zin.
Parentage:
   Female and male parent.—‘Mahogany Splendor’, not patented.
Propagation:
   Type cutting.—Terminal stem.
   Time to initiate roots.—Approximately 6 to 8 days.
   Time to produce a rooted cutting.—Approximately 21 to 28 days.
   Root description.—Fibrous, white to light brown in color.
   Rooting habit.—Freely branching.
Plant Description:
   Commercial crop time.—Approximately 8 to 10 weeks from a rooted cutting to finish in a 15 cm pot.
   Growth habit and general appearance.—Moderately vigorous, upright-mounded.
   Size.—Height: Approximately 78.0 cm. Width: Approximately 50.0 cm.
   Branching habit.—Freely branching, pinching enhances basal branching. Quantity of main lateral branches per plant: Approximately 6.
   Branch.—Strength: Strong. Length: Approximately 21.0 cm. Diameter: Approximately 3.0 mm. Length of central internode: Approximately 5.5 cm. Texture: Sparsely glandular pubescent. Gland color: Colorless, transparent. Color of young stems: 146A with an overlay of 187A. Color of mature stems: 146A with a heavy overlay of 187A.
Foliage description:
   General description.—Quantity of leaves per main branch: Approximately 7. Fragrance: None detected. Form: Simple. Arrangement: Alternate, spiral.
   Leaves.—Shape: Orbicular in overall form, palmately divided with 3 or 5 lobes. Margin: Crenate. Apex of lobes: Rounded. Base: Obtuse to truncate. Venation pattern: Palmate. Length of mature leaf: Approximately 10.0 cm. Width of mature leaf: Approximately 12.0 cm. Texture of upper and lower surfaces: Sparsely glandular pubescent. Gland color: Colorless, transparent. Color of upper surface of young foliage: 146A and 137A with a faint overlay and midveins of 187B. Color of lower surface of young foliage: Closest to 147B with midveins of 184A. Color of upper surface of mature foliage: N137A to N137B with an overlay and midveins of 187A, overlay increases in intensity under outdoor conditions so that foliage appears burgundy colored. Color of lower surface of mature foliage: Between 147A and 147B with midveins of 184A.
   Petiole.—Length: Approximately 8.5 cm. Diameter: Approximately 2.0 mm to 3.0 mm. Texture: Moderately pubescent with a mixture of glandular and nonglandular hairs. Gland color: Colorless, transparent. Color: 146A with 137A and a heavy overlay of 187A.
Flowering description:
   Flowering habit.—‘Little Zin’ is grown for its foliage interest; however, it flowers sporadically under outdoor growing conditions from spring through late spring and year-round in greenhouse environment.
   Lastingness of individual flower on the plant.—Flowers open at a rate of one per plant, and were observed for approximately two to three hours in the morning.
Flower description:
   General description.—Type: Solitary, not persistent. Shape: Round. Aspect: Facing upward and outward. Arrangement: In leaf axils. Appearance: Velvety. Fragrance: None detected.
   Bud.—Rate of opening: Generally takes less than 24 hours for bud to progress from first color to fully open flower. Quantity per plant: Approximately 2.
   Bud just before opening.—Shape: Ovoid. Length: Approximately 1.6 cm. Diameter: Approximately 1.0 cm. Texture: Densely glandular pubescent. Gland color: Colorless, transparent. Color: 146A with carina of 187A surrounded by 145C.
   Corolla.—Diameter: Approximately 7.0 cm. Depth: Approximately 2.5 cm.
   Petals.—Quantity: 5, non-imbricate. Shape: Obovate. Margin: Entire, slightly ruffled appearance. Apex: Obtuse. Base: Claw-like. Length: Approximately 4.0 cm. Width: Approximately 2.5 cm. Texture of upper surface: Glabrous, velvety with impressed venation. Texture of lower surface: Upper third tomentose, ribbed venation. Color of upper surface when fully open: Closest to 187A with venation of 187B, dark ring of 186A approximately 3.0 cm in diameter. Color of lower surface when fully open: Closest to 187A with venation of 187B.
   Calyx.—Diameter: Approximately 3.0 cm. Depth: Approximately 1.9 cm.
   Sepals.—Quantity per flower: 5, fused at base. Shape: Lanceolate. Apex: Sharply acute. Length: Approximately 1.9 cm. Width: Approximately 6.0 mm. Texture of upper (inner) surface: Glabrous. Texture of lower (outer) surface: Densely glandular pubescent. Gland color: Colorless, transparent. Color of upper (inner) surface: Closest to 146C. Color of lower (outer) surface: 146A with carina in the center of each sepal of 187A surrounded by 145C.
   Epicalyx.—Appearance: Single whorl subtends calyx, fused at base. Diameter: Approximately 3.0 cm. Depth: Approximately 1.7 cm.
   Bracts.—Quantity per flower: Approximately 9. Shape: Lanceolate blade attached to a stalk. Apex: Acute. Length: Total length approximately 1.7 cm of which blade portion is 8.0 mm. Width: Blade portion approximately 4.0 mm and stalk approximately 2.0 mm. Texture of upper and lower surfaces: Sparsely glandular pubescent. Gland color: Colorless, transparent. Color of upper (inner) surface: 137A with an overlay of 187A on blade portion. Color of lower (outer) surface: 137A with an overlay of 183A on blade portion, base of 144A.
   Peduncle.—Strength: Strong. Aspect: Acute angle to stem. Length: Approximately 2.0 mm. Diameter: Approximately 1.5 mm. Texture: Densely glandular pubescent. Gland color: Colorless, transparent. Color: 137B.
   Reproductive organs.—Androecium: Stamen quantity: Approximately 25 per flower, fused to a staminal column surrounding the style. Stamen length: Approximately 3.0 mm. Filament length: Approximately 2.0 mm. Filament color: Close to 187A quickly darkening to N186A. Anther shape: Oblong, arched, filament attached at peak of arch. Anther length: Approximately 2.0 mm. Anther color: 162A quickly darkening to N186A. Pollen amount: Abundant. Pollen color: 162A quickly darkens to 164B and 165B. Gynoecium: Pistil quantity: One per flower, style five-branched. Pistil length: Approximately 2.4 cm. Stigma shape: Rounded. Stigma length: Approximately 1.0 mm. Stigma color: 187A. Style length: Approximately 1.7 cm, including 2.0 mm style branches. Style color: 145B at base transitioning through 187C to style branches of 187B. Ovary length: Approximately 6.0 mm. Ovary color: 4D.
Seed and fruit production: Neither seed nor fruit production has been observed.
Disease and pest resistance: Resistance to pathogens and pests common to Hibiscus has not been observed.

What is claimed is:

1. A new and distinct cultivar of Hibiscus plant named ‘Little Zin’, substantially as herein illustrated and described.
